(19) United States
(12) Plant Patent Application Publication
(10) Pub. No.: US 2023/0337560 P1
(43) Pub. Date: Oct. 19, 2023

(54) CANNABIS PLANT NAMED 'OW-1'

(50) ;

(71) Applicants: David Straub, Willow, AK (US); Matt Gore, Wasilla, AK (US)

(72) Inventors: David Straub, Willow, AK (US); Matt Gore, Wasilla, AK (US)

(21) Appl. No.: 18/445,004

(22) Filed: Feb. 27, 2023

Related U.S. Application Data
(63) Continuation-in-part of application No. 17/119,082, filed on Dec. 11, 2020, now abandoned.
(60) Provisional application No. 62/947,519, filed on Dec. 12, 2019.

(51) Int. Cl. A01H 6/28

(52) U.S. Cl. PLT/258

(57) ABSTRACT

The unique Cannabis plant variety C. sativa ‘OW-1’ is provided. The variety can be distinguished by its elevated levels of CBGA.

### CROSS REFERENCE TO RELATED APPLICATIONS

The present Application for Patent is a continuation-in-part of U.S. application Ser. No. 17/119,082 filed on Dec. 11, 2020, which in turn claims the benefit of provisional application No. 62/947,519 entitled “CANNABIS PLANT NAMED ‘OW-1’” filed Dec. 12, 2019, and hereby expressly incorporated by reference herein.

Latin name of the genus and species: Genus—Cannabis. Species—sativa.

### VARIETY DENOMINATION

The new Cannabis plant claimed is of the variety denominated ‘OW-1’.

### BACKGROUND OF THE INVENTION

### Field of the Invention

The present invention relates to a new and distinct annual variety of C. sativa hybrid, which has been given the variety denomination of ‘OW-1’. ‘OW-1’ is intended for use as medicinal herb, for sale in Cannabis dispensaries, and as a home garden plant.

### Background of the Related Art

The genus Cannabis has been in use by humans for millennia, due to the multiplicity of its benefits to humans, including the considerable value and utility of its fiber, the nutritional value of its seeds, and the medicinal value of its floral parts and products made from them. Currently the genus is under intense legal commercialization in the United States as industrial hemp for a variety of purposes including biodegradable plastics and building materials, clothing, paper, food, fuel and medicines.

Cannabidiol (CBD) extracted from Cannabis is widely used in over-the-counter medicines and topical treatments and is also the active ingredient in the FDA-approved drug Epidiolex. CBD is just one of at least dozens—perhaps hundreds—of cannabinoids endogenous to Cannabis, tetrahydrocannabinol (THC) being the other cannabinoid that is most well-known. The cannabinoids as a group interact with the human endocannabinoid receptors, which are distributed in the brain and throughout the body. The study of the endocannabinoid system (ECS) in humans and other mammals is an area of increasing interest and holds tremendous promise for the future of medicine. See, e.g., Russo (2019). Cannabis and Pain, Pain Medicine, 20(10): 1093/pm/pnz227; and Russo (2016). Clinical Endocannabinoid Deficiency Reconsidered: Current Research Supports the Theory in Migraine, Fibromyalgia, Irritable Bowel, and Other Treatment-Resistant Syndromes, Cannabis Cannabinoid Res. 1(1): 154-165.

Non-hemp forms of Cannabis, frequently referred to as marijuana, have been legalized for medicinal use in many states and also for recreational use (sometimes called “adult use”) in a growing number of states including Alaska, Arizona, California, Colorado, Illinois, Maine, Massachusetts, Michigan, Montana, New Jersey, Nevada, Oregon, Vermont, and Washington. It is expected that the wave of legalization will continue to the point of some form of federal legalization or decriminalization.

Typically, marijuana products are available to users for purchase in specialized “dispensaries” that offer dried flower, edibles, tinctures, extracts, and the like. In some cases, a unique or unusual chemical profile, or chemotype, is attractive not only for flower sales but also for use in the preparation of extracts and/or isolates and for the manufacture of a variety of products that possess characteristics of the chemotype.

### SUMMARY OF THE INVENTION

C. sativa cultivar ‘OW-1’ is a new cultivar has elevated levels of CBGA. C. sativa cultivar ‘OW-1’ is a tall growing plant. The variety has a strong fragrance at 7 weeks old. The variety has a very low flower to leaf ratio of 6 to 1. The variety grows best with warm days, cool nights, and high nutrient base feeding.

The variety can be used to treat certain medical conditions. For example, the variety can be used to treat rheumatoid arthritis. For example, the variety can be used to treat bowel disorders such as Crohn's Disease.

The genetics of the plant are wild Oaxacan and a cultivar known as ‘Pennywise.’

Methods of growing the variety include the use of soil comprising high calcium magnesium. The soil can have a pH of 6.0-6.5.

The variety has been asexually reproduced for over 2 years in Willow, Alaska.

‘OW-1’ can have a cannabinoid profile as set forth in Table 1 and a terpene profile as set forth in Table 2. The chemotypic characteristics of the new variety are variable based upon cultivation conditions, as is typical of Cannabis plants.

TABLE 1
Exemplary Profiles of Key Cannabinoids.

| | Percent | Percent | Percent | | |
|---|---|---|---|---|---|
| THCA | 12.54 | 16.90 | 21.80 | 26.16 | 30.52 |
| THC | 0.02 | 0.18 | 0.79 | 1.83 | 1.99 |
| Total THC* | 12.24 | 16.23 | 19.95 | 23.70 | 27.69 |
| THCVA | 0.00 | 0.27 | 0.05 | 0.23 | 0.24 |
| CBGA | 0.00 | 1.90 | 4.20 | 6.15 | 6.99 |
| CBG | 0.07 | 0.17 | 0.52 | 0.71 | 0.81 |
| Total CBD | 0.03 | 0.04 | 0.06 | 0.07 | 0.08 |
| Total | 15.18 | 20.67 | 27.47 | 32.22 | 37.71 |
| Cannabinoid | | | | | |

*Total THC = (THCA * 0.877) + THC (i.e. delta 9 THC) + delta 8 THC

TABLE 2
Exemplary Profiles of Key Terpenes.

| | Percent | Percent | Percent | Percent | Percent |
|---|---|---|---|---|---|
| Alpha Pinene | 0.04 | 0.06 | 0.11 | 0.31 | 0.33 |
| P Cymene | 0.00 | 0.01 | 0.01 | 0.09 | 0.09 |
| G-Terpinene | 0.01 | 0.02 | 0.03 | 0.04 | 0.05 |
| A-Humulene | 0.00 | 0.02 | 0.13 | 0.25 | 0.28 |
| A-Bisabolol | 0.00 | 0.01 | 0.01 | 0.02 | 0.02 |
| Beta Pinene | 0.07 | 0.09 | 0.13 | 0.18 | 0.21 |
| D3carene | 0.03 | 0.04 | 0.07 | 0.10 | 0.11 |
| Limonene | 0.11 | 0.14 | 0.17 | 0.25 | 0.28 |
| Terpinolene | 0.00 | 0.32 | 0.64 | 1.08 | 1.21 |
| Neroliodol | 0.00 | 0.01 | 0.03 | 0.04 | 0.05 |
| Beta Myrcene | 0.21 | 0.30 | 0.47 | 0.80 | 0.89 |
| A-Terpinene | 0.00 | 0.01 | 0.02 | 0.05 | 0.05 |
| Ocimene | 0.27 | 0.39 | 0.60 | 1.05 | 1.17 |
| Linalool | 0.01 | 0.01 | 0.02 | 0.06 | 0.06 |
| B-Caryophyllene | 0.06 | 0.14 | 0.38 | 0.68 | 0.76 |
| Terpene Total | 0.96 | 1.56 | 3.01 | 4.80 | 5.40 |

### BRIEF DESCRIPTION OF PICTURES

The accompanying photographs show the typical appearance of the new variety ‘OW-1’. The colors are as nearly true as is reasonably possible in a color representation of this type. Colors in the photographs may differ slightly from the color values cited in the detailed botanical description which accurately describes the colors of the new plant.

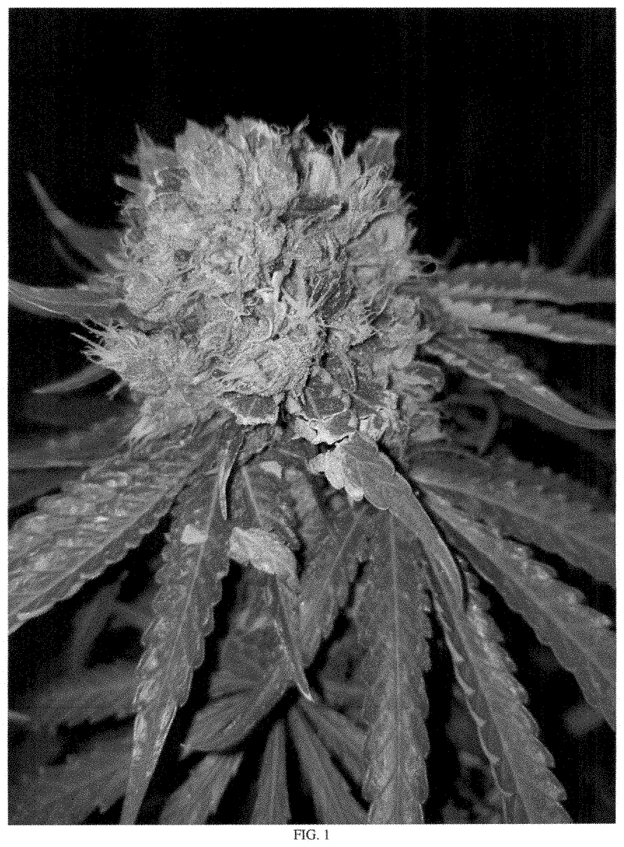

FIG. 1 depicts inflorescence of ‘OW-1’.

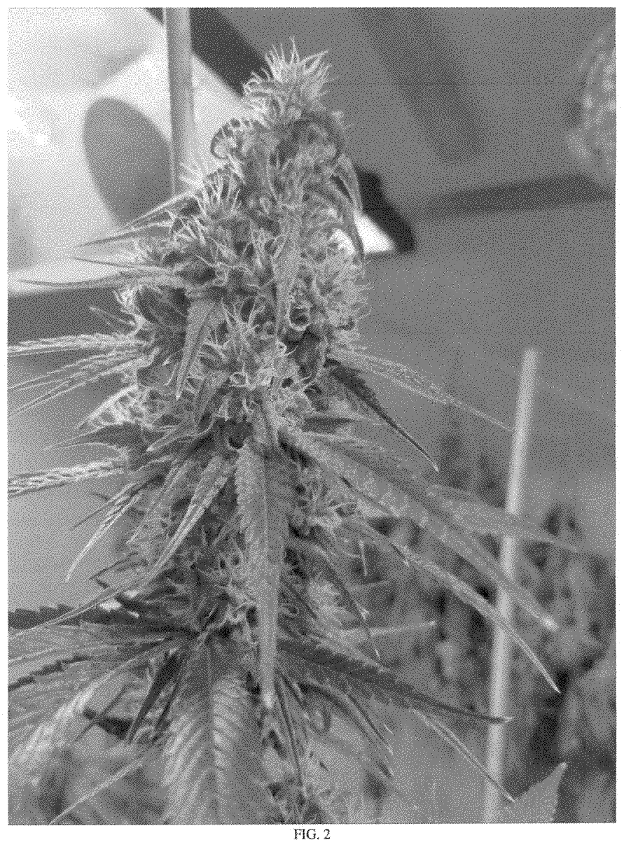

FIG. 2 depicts inflorescence of ‘OW-1’.

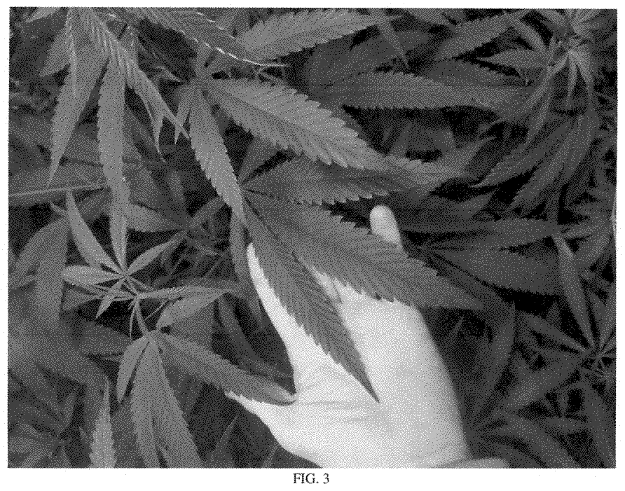

FIG. 3 depicts the leaves of ‘OW-1’.

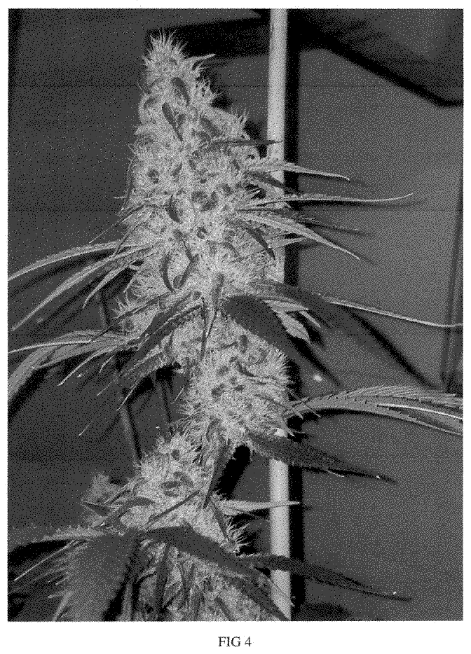

FIG. 4 depicts inflorescence of ‘OW-1’.

### DETAILED BOTANICAL DESCRIPTION

The following detailed description sets forth the distinctive characteristics of ‘OW-1’. The data which define these characteristics were collected from asexual reproductions of the original selection. Dimensions, sizes, colors, and other characteristics are approximations and averages set forth as accurately as possible.
Type: Herbaceous tap-rooted annual.
   a. Family.—Cannabaceae.
   b. Genus.—Cannabis.
   c. Species.—sativa.
   d. Common name.—Marijuana.
Market class: A medicinal herb intended for use as medical oil, and medicinal herb for sale in Cannabis dispensaries and as a home garden plant.

1. A new and distinct variety of Cannabis plant named ‘OW-1’, as illustrated and described herein.
